(19) United States
(12) Patent Application Publication
(10) Pub. No.: US 2007/0161981 A1
(43) Pub. Date: Jul. 12, 2007

(54) Electrosurgical method and systems for treating glaucoma

(75) Inventors: Norman R. Sanders, Hillsborough, MD (US); Jean Woloszko, Mountain View, CA (US); Robert H. Dahla, Sunnyvale, CA (US)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/327,553

(22) Filed: Jan. 6, 2006

(51) Int. Cl. A61B 18/14

(52) U.S. Cl. 606/41; 606/32

(57) ABSTRACT

An electrosurgical method for treating open angle and narrow angle glaucoma, comprising positioning an active electrode in close proximity to a drainage angle of the eye, the active electrode disposed on a distal end of a shaft; and applying a high frequency voltage difference between the active electrode and a return electrode sufficient to ablate and coagulate target tissue in the vicinity of the drainage angle, to create drainage canals with prolonged patency.

### BACKGROUND

### Field of Invention

This invention pertains to an electrosurgical method and system for treating glaucoma; in particular, an plasma-mediated method and system for treating narrow angle glaucoma whereby drainage canals are created within the iris to facilitate fluid drainage in the eye; and a plasma-mediated method and system for treating open angle glaucoma whereby small defects with self-sealing borders are created in the iris for prolonged patency to facilitate fluid drainage and reduce intraocular pressure in the eye.

### SUMMARY OF THE INVENTION

In one embodiment, the present method is a procedure for treating both open angle (OAG) and narrow angle glaucoma (NAG), comprising positioning an active electrode in close proximity to the drainage angle of the eye, the active electrode disposed on a distal end of a shaft; and applying a high frequency voltage difference between the active electrode and a return electrode sufficient to ablate and coagulate target tissue in the vicinity of the drainage angle, to open and create drainage canals, with prolonged patency. In one embodiment, the method creates drainage canals in the trabecular meshwork between the iris and the Schlemm canal in the eye to relieve the symptoms of OAG; in particular, the drainage canals are formed with stabilized borders that resist scar formation and closure. In another embodiment, the method creates a small defect in the vicinity of the drainage angle of the eye to relieve the symptoms of NAG; in particular, the defect are formed with self-sealing borders for prolonged patency. In both embodiments, the procedures result in creating pathways for relieving excess intraocular pressure.

In various embodiments, plasma is generated at the electrode of the electrosurgical apparatus in the presence of a conductive fluid by suitably adjusting the voltage to ablate tissue in the drainage angle. Also, by suitably adjusting the voltage, the method and system coagulate tissue in the target location to create the stabilized borders that resist fibrous tissue formation and form the self-sealing borders around a small defect for prolonged patency.

Embodiments of the present method and system are illustrated in the following Figures, and are described in detail in the following specifications.

### BRIEF DESCRIPTION OF THE DRAWINGS

FIG. 1 is an illustration of a cross-section of a human eye.

FIG. 2A is an illustration of normal fluid flow in the human eye.

FIG. 2B is an illustration of fluid flow in the eye associated with open-angle glaucoma.

FIG. 2C is an illustration of fluid flow in the eye associated with narrow angle glaucoma.

FIG. 3 is an illustration of an electrosurgical system for treating open-angle and narrow angle glaucoma in accordance with the present method.

FIGS. 4A-4H are illustrations of an electrosurgical apparatus and electrode configurations for treating narrow-angle and open-angle glaucoma in accordance with the present method.

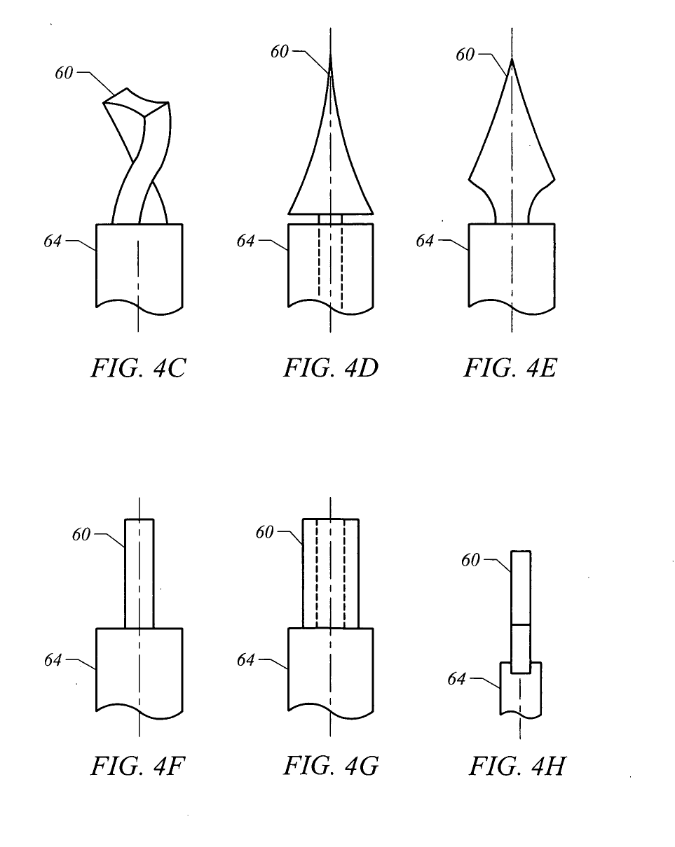

FIG. 3B is an illustration of an electrode assembly for treating narrow-angle and open-angle glaucoma in accordance with the present method.

FIG. 5 is an algorithm of an embodiment of the present method.

### DETAILED DESCRIPTION

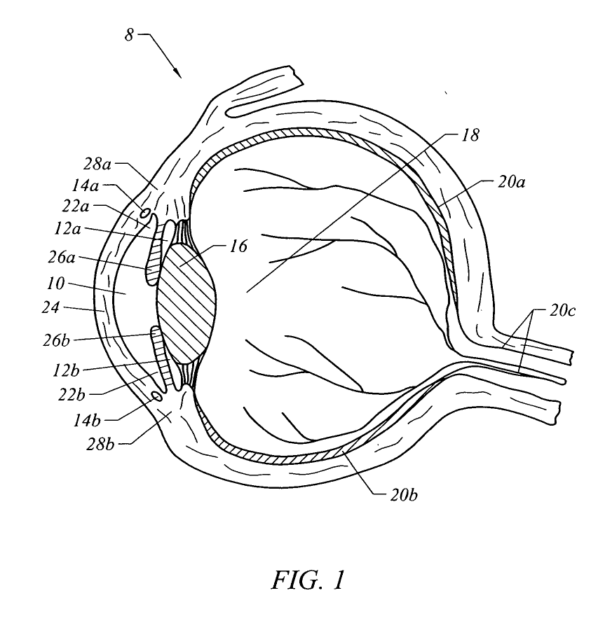

With reference to FIG. 1, glaucoma is a group of eye diseases linked to deterioration or damage to the optic nerve (20c) in the retina (20a, 20b). If the condition is not treated the deterioration may lead to visual field loss and blindness. In the human eye, the optic nerve transmits visual images to the brain; if the nerve is damaged, the transmission of the images to the brain is disrupted. One factor that causes damage to the optic nerve is an increase in the intraocular pressure (IOP) in the eye; however the damage may also be due to other causes such as vascular insufficiency.

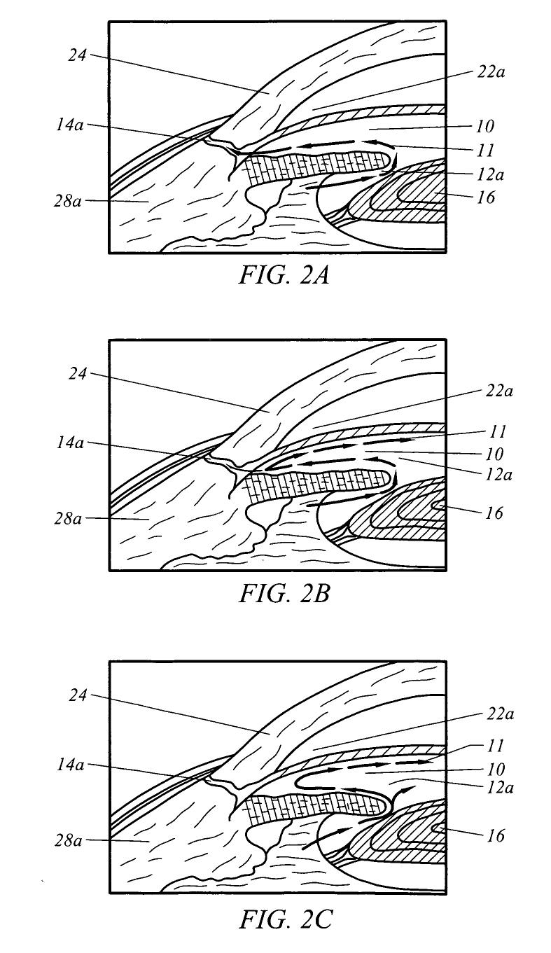

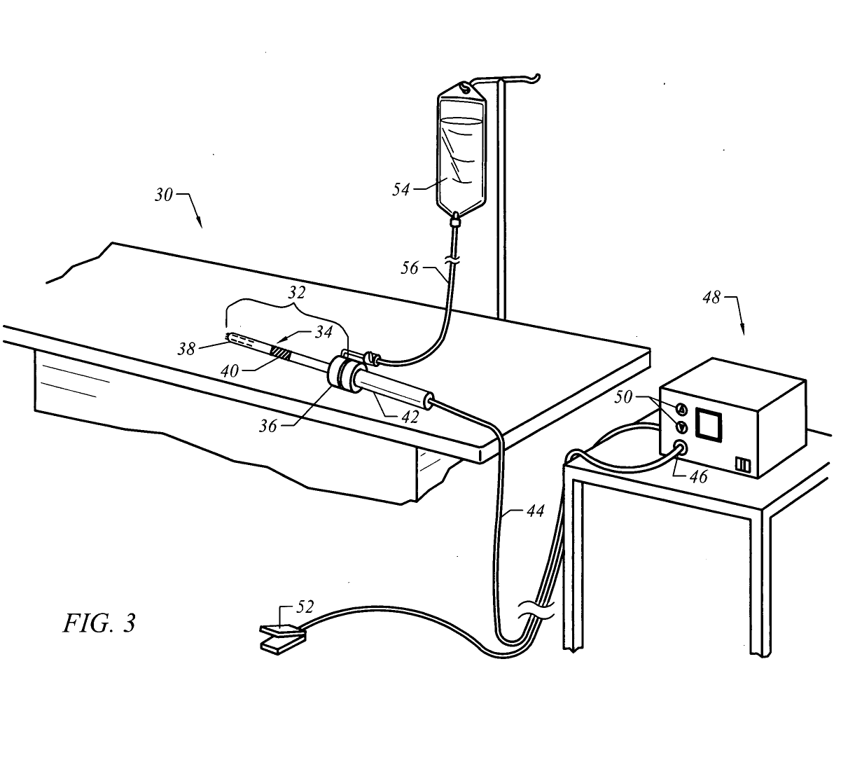

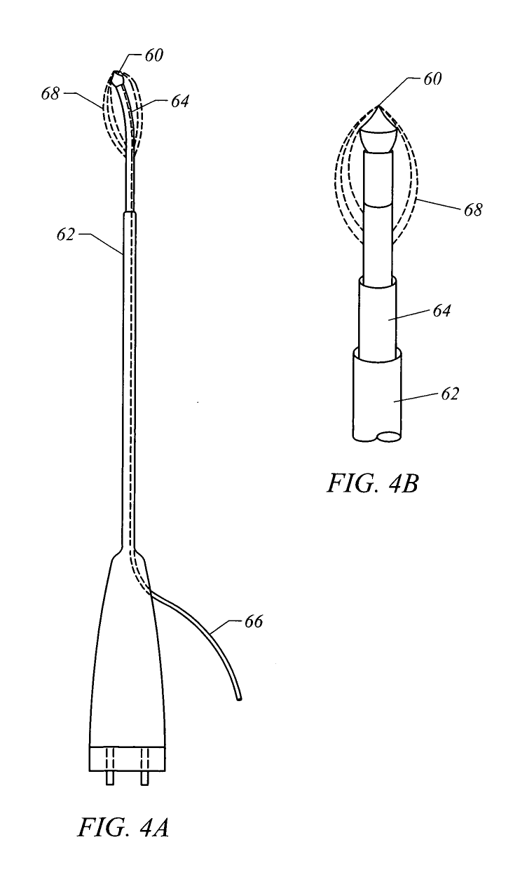

Referring to FIGS. 1, 2A, 2B and 2C wherein sections of the human eye (8) are illustrated, in a normally functioning eye, aqueous humor is produced by the ciliary body to nourish the anterior chamber (10) and the posterior chambers (12a, 12b). Excess amounts of humor from the chambers are drained primarily through a network of canals in the trabecular meshwork located in the drainage angle of the eye (22a, 22b), into the Schlemm's canal (14a, 14b) from where it is drained into the veins. Drainage of excess humor, shown illustratively by the arrows (11) in FIG. 2A helps to maintain a healthy level of IOP, normally between 12 mm and 20 mm of mercury. The drainage angle (22a, 22b) is that portion of the eye located at the confluence of the eye's clear covering (the cornea (24)), the eye's colored part (the iris (26a, 26b)) and where the iris meets the white outer covering of the eye (the sclera (28, 28b)).

With reference to FIG. 2B and 2C, if drainage of aqueous humor (11) from the chambers (10, 12a, 12b) is restricted as is illustrated n FIG. 2B, or completely blocked as in FIG. 2C, the fluid pressure in the chambers increase, which in turn increases the pressure throughout the interior of the eye. With the increase in pressure in the chambers (10, 12a, 12b), the pressure on the lens (16), the vitreous fluid chamber (18) and the retina (20a, 20b) at the back of the eye containing the optic nerves (20c) is increased. If the increased pressure on the retina persists for extended periods, the vessels to the axons and neurons of the optic nerves are compressed, resulting in damage to the optic nerve. While not all instances of an elevated IOP will cause glaucoma, patients with an elevated IOP are at a greater risk for developing the condition.

With reference to FIGS. 2B and 2C, glaucoma is categorized into two broad classifications, OAG and NAG, based on the location of the restriction (or blockage) that causes the elevated IOP. For example, with reference to FIG. 2B, the OAG condition results where there is a restriction or blockage within the drainage canals of the trabecular meshwork (22a). As the restriction or blockage prevents excess humor in the chambers (10, 12a) from passing through the trabecular meshwork into the Schlemm canal (14a), the fluid pressure in the chambers raises which, in turn, raises the pressure throughout the interior of the eye as described above. With OAG, a relatively small amount of fluid may pass into the Schlemm canal as is illustrated in FIG. 2B, in which case the increase in the IOP rises relatively slowly; also, with OAG the restricted fluid flow may occur in both eyes at about the same time, although in some patients one eye may be more severely affected than the other.

With regard to the restriction or blockage that causes NAG, as is illustrated in FIG. 2C, this condition usually occurs when the drainage angle (22a) between the iris and the cornea (24) is too small, and the iris moves over to cover and block the drainage angle, and thus block the access to the drainage canals in the trabecular meshwork. With this blockage, excess fluid in the chambers (10, 12a) is prevented from draining into the canals of the trabecular meshwork and consequently the IOP rises. The blockage is exacerbated on patients with a small anterior chamber (10) that provides a smaller drainage angle for the aqueous humor to pass through. As excess fluid (11) builds up behind the iris in the trabecular meshwork, the pressure further narrows the angle. Also, on some patients with NAG, because the angle between the iris and cornea is not as wide and as open as it should be, the outer edges of the iris (26a, 26b) bunches-up over the drainage canals when the pupil enlarges either too much or too quickly. The bunching-up can occur, for example, on entering a dark room, which causes the internal pressure to increase. On patients with NAG since the fluid is prevented from draining into the Schlemm canal as is illustrated in FIG. 2C, the IOP can increase rapidly to cause vision loss in just a few days after diagnosis.

Conventional treatments to relieve glaucoma due to elevated IOP vary, depending on the cause of the condition. Treatment includes eye-drop medication and or surgery to lower the IOP. Both medication and surgery treatments attempt to drain fluids from the eye and lower the IOP and/or decrease the amount fluid flowing into the eye. With surgery, various procedures are utilized including laser trabeculoplasty, trabeculectomy (or filtering microsurgery), and trabeculectomy with implant, each, however, with mixed results. With laser trabeculoplasty, for example, the eye is numbed and the laser beam is aimed into the eye through a special lens that makes a camera-like flash into the eye to open the drainage angle. Laser trabeculoplasty improves fluid drainage by burning tissue and causing scarring, to open-up canals in the trabecular meshwork. The opened canals make it easier for fluids to flow out and in the front part of the eye, to decrease the IOP. However, if excessive scar tissue forms, further surgery may be needed. With filtering microsurgery, a tiny drainage hole is made in the sclera to allow fluid to flow out of the eye and lower the IOP.

A problem in treating glaucoma with conventional procedures is that, flowing treatment, scar tissue tends to form and obstruct fluid flows to and through the drainage canals. In particular, with prior procedures for treating NAG, fibrous scar tissue formation and closure of the drainage canal is a common, while with OAG, the re-closure of the opening over the drainage canals is common.

In accordance with the present method and system, these problems are addressed by a method wherein in one embodiment, NAG is treated using an electrosurgical apparatus to create drainage canals within the iris to facilitate fluid drainage in the eye. In this embodiment the drainage canals are formed with stabilized borders that resist fibrous scar formation and thereby avoid the problem of re-closure after the procedure. In another embodiment, using the electrosurgical apparatus, NAOG is treated by creating small openings or defects with self-sealing borders in the iris, for prolonged patency to facilitate fluid drainage.

In one embodiment, a system and apparatus for treating OAG and NAG in accordance with the present procedure is illustrated in FIG. 3. Such a system is described in further detail in commonly owned U.S. Pat. Nos. 6,296,638, 6,602,248 and 6,805,130 the disclosures of which are herein incorporated by reference for the present purposes. In the embodiment illustrated in FIG. 3, the system (30) comprises an electrosurgical apparatus that includes a probe (32) comprising an elongated shaft (34) and a connector (36) at its proximal end, and one or more active electrodes (38) disposed on the distal end of the shaft. Also disposed on the shaft but spaced from the active electrode is a return electrode (40). The probe includes a handle (42) with connecting power cable (44) and cable connector (46) that can be removably connected to the power supply (48).

As used herein an active electrode is an electrosurgical electrode, as described for example in commonly owned U.S. Pat. Nos. 6,296,638, 6,602,248 and 6,805,130 incorporated by reference, that are adapted to generate a higher charge density, and hence generate more plasma, relative to a return electrode when a high-frequency voltage potential is applied across the electrodes. Typically, a higher charge density is obtained by making the active electrode surface area smaller relative to the surface area of the return electrode.

Continuing with reference to FIG. 3, the present system includes a power supply (48) that comprises selection switches (50) to change the applied voltage level. In various embodiments, the power supply (48) can also include a foot pedal (52) positioned close to the user for energizing the electrodes (38, 40). The foot pedal (52) may also include a second pedal (not shown) for remotely adjusting the voltage level applied to electrodes (38,40). Also included in the system is an electrically conductive fluid supply (54) with tubing (56) for supplying the probe (32) and the electrodes with electrically conductive fluid. Details of a power supply that may be used with the electrosurgical probe of the present invention is described in commonly owned U.S. Pat. No. 5,697,909 which is hereby incorporated by reference herein.

As is illustrated in FIGS. 3, in one embodiment the return electrode (40) is connected to power supply (48) via cable connectors (44), to a point slightly proximal of active electrode.

Typically the return electrode is spaced at about 0.5 mm to 10 mm, and more preferably about 0.5 mm to 3 mm from the active electrode. Shaft (34) is disposed within an electrically insulative jacket, which is typically formed as one or more electrically insulative sheaths or coatings, such as polyester, polytetrafluoroethylene, polyimide, polyethylene and the like. The electrically insulative jacket over shaft (34) prevents direct electrical contact between shaft (34) and any adjacent body structure or the operator.

As will be appreciated by one ordinarily skilled in the art, the above-described systems and apparatus can be used equally well in a wide range of electrosurgical procedures to treat body tissue including open procedures, intravascular procedures, urological, laparoscopic, arthroscopic, thoracoscopic or other cardiac procedures, as well as dermatological, orthopedic, gynecological, otorhinolaryngological, spinal, and neurologic procedures, oncology and the like.

However, for the present purposes the system described herein is directed to treating various forms of glaucoma, including NAG and OAG glaucoma.

In accordance with the present method, the system of FIG. 3 is adapted to apply a high frequency (RF) voltage/current to the active electrode(s) to modify the structure of tissue on and in the vicinity of the trabecular meshwork in the drainage angle. In one embodiment an electrically conductive fluid is present and is in contact with at least the active electrode. The electrically conductive fluid includes isotonic saline, a conductive gel, extra-cellular fluid and other body fluids such as blood, aqueous based body fluid such as eye tears. In one embodiment for treating OAG with the present method, the system of FIG. 3 is set to a relatively higher voltage suitable for cobalting tissue. At the higher voltage, the active electrode is used to create drainage canals in the trabecular meshwork by creating perforations in the drainage angle by volumetrically removing tissue in the drainage angle (i.e., ablate or effect molecular dissociation of the tissue structure) within the trabecular meshwork. Thereafter, at a lower voltage level suitable for coagulating tissue, the canals are treated with the active electrode to form stabilized borders that resist fibrous scar formation and closure. At the lower voltage level it is believed that contraction and shrinkage of collagen-containing connective tissue and severed blood vessels in and around the trabecular meshwork contribute to the formation of the stabilized borders.

Similarly in treating NAG, the system of FIG. 3 is adapted to apply a high frequency (RF) voltage/current to the active electrode(s) to modify the structure of tissue on and in the vicinity of the trabecular meshwork. In one embodiment an electrically conductive fluid is present and is in contact with at least the active electrode. The electrically conductive fluid includes isotonic saline, a conductive gel, intra-cellular fluid and other body fluid such as blood and eye tears. In treating OAG with the present method, the system of FIG. 3 is used to create a small defect that opens the iris into the trabecular meshwork. As with the procedure for treating NAG, the defects are created with a self- sealing border to assure prolonged patency through the iris and the trabecular meshwork. In one embodiment the defect is created by: perforating the drainage angle and coagulating tissue around the opening, the coagulated tissue serving to prolong the patency of the opening. In this procedure, tissue in the drainage angle may be volumetrically removed or destroyed (i.e., ablated to effect molecular dissociation of the tissue structure) within the trabecular meshwork to form holes, channels, divots, or other spaces on the trabecular meshwork. Also, by adjusting the voltage across the electrodes the tissue may be coagulated or shrunk by contracting collagen-containing connective tissue in and around the trabecular meshwork. The voltage may also be adjusted to coagulate severed blood vessels in and around the trabecular meshwork to stop bleeding.

In accordance with the present method, the high frequency voltage difference applied between one or more active electrode(s) and one or more return electrode(s) on the electrosurgical apparatus develop high electric field intensities and plasma in the vicinity of the target tissue. The high electric field intensities adjacent to the active electrode(s) induces molecular breakdown of target tissue by molecular dissociation of tissue components (rather than by thermal evaporation or carbonization). In this procedure it is believed that the tissue structure is volumetrically removed by molecular disintegration of larger organic molecules into smaller molecules and/or atoms, such as hydrogen, oxygen, oxides of carbon, hydrocarbons and nitrogen compounds, by the plasma. This molecular disintegration completely removes the tissue structure, as distinct from dehydrating the tissue material by the removal of water from within the cells of the tissue, as with other non-plasma procedures.

The high electric field intensity used in the present method is generated by applying a high frequency voltage that is sufficient to vaporize electrically conductive fluid disposed over at least a portion of the active electrode(s) in the region between the distal tip of the active electrode(s) and the target tissue. The electrically conductive fluid may be a liquid, such as isotonic saline, extra-cellular fluid, ringer lactate solution, blood and other body fluids delivered to the target site, or a viscous fluid, such as a gel, applied to the target site. Since the vapor layer or vaporized region has relatively high electrical impedance, it minimizes current flow into the electrically conductive fluid. This ionization, under these conditions, induces the discharge of plasma comprised of energetic electrons and photons from the vapor layer and to the surface of the target tissue. A more detailed description of this phenomenon, termed Coblation™, can be found in commonly assigned U.S. Pat. No. 5,683,366 the complete disclosure of which is incorporated herein by reference.

In various embodiments of the present method, the electrically conductive fluid possesses an electrical conductivity value above a minimum threshold level, in order to provide a suitable conductive path between the return electrode and the active electrode(s). The electrical conductivity of the fluid (in units of milliSiemens per centimeter or mS/cm) is usually be greater than about 0.2 mS/cm, typically greater than about 2 mS/cm and more typically greater than about 10 mS/cm. In an exemplary embodiment, the electrically conductive fluid is isotonic saline, which has a conductivity of about 17 mS/cm.

Also in various embodiments of the preset method, it may be necessary to remove, e.g., aspirate, any excess electrically conductive fluid and/or ablation by-products from the surgical site. In addition, it may be desirable to aspirate small pieces of tissue that are not completely disintegrated by the high frequency energy, or other fluids at the target site, such as blood, mucus, and other body fluids.

In one embodiment, the present system includes one or more suction lumen(s) in the shaft, or on another instrument, coupled to a suitable vacuum source for aspirating fluids from the target site. In various embodiments, the instrument also includes one or more aspiration electrode(s) coupled to the aspiration lumen for inhibiting clogging during aspiration of tissue fragments from the surgical site. A more complete description of these embodiments can be found in commonly owned U.S. Pat. No. 6,190,381, the complete disclosure of which is incorporated herein by reference for all purposes.

In one embodiment of the present method, a single electrode or an electrode array may be disposed over a distal end of the shaft of the electrosurgical instrument to generate and apply plasma to the tissue. In either configuration, the circumscribed area of the electrode or electrode array will generally depend on the desired diameter of the perforations and amount of tissue to be removed. In one embodiment, the diameter of the electrode array is in the range of from about 0.25 mm to 20 mm, preferably from about 0.25 mm to 10 mm, and more preferably from about 0.25 mm to 0.3 mm.

In addition, the shape of the electrode at the distal end of the instrument shaft will also depend on the size of the surface area to be treated. For example, the electrode may comprise a pointed tip, a round wire, or a wire having other solid cross-sectional shapes such as squares, rectangles, hexagons, triangles, star-shaped, or the like, to provide a plurality of edges around the distal perimeter of the electrodes. Alternatively, the electrode may comprise a hollow metal tube having a cross-sectional shape that is round, square, hexagonal, rectangular or the like. The envelope or effective diameter of the individual electrode(s) ranges from about 0.05 mm to 3 mm, preferably from about 0.1 mm to 2 mm.

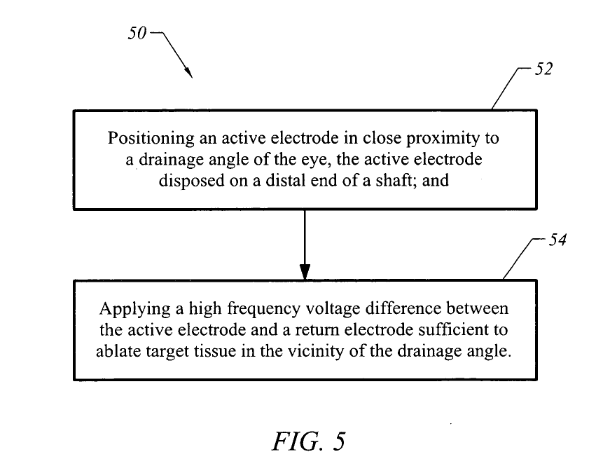

Examples of electrosurgical apparatus that can be used to ablate and modify tissue in accordance with the present method are illustrated in FIG. 4A with enlarged portions of suitable electrode tips shown in FIGS. 4b-4h. In one embodiment the apparatus comprises an active electrode (60) disposed on the distal end of a shaft (62). Spaced from the active electrode is a return electrode (64) disposed on the shaft. In a preferred embodiment illustrated in FIG. 4c, the active electrode tip comprises a twist drill having a diameter in the range of 0.20 mm to 0.711 mm that correspond to nominal twist drill # 92 to 70. In all embodiments illustrated both the active and return electrodes are connected to a high frequency voltage supply (not shown). Disposed in contact with the active and return electrodes is an electrically conductive fluid supply (66). In one embodiment the electrically conductive fluid supply forms an electrically conductive fluid bridge (68) between the electrodes. On application of a high frequency voltage across the active and return electrode, plasma is generated as described above, for use in accordance with the present method. A more detailed description of this phenomenon, termed Coblation™, and the operation of the electrode illustrated in FIG. 4A and 4B be found in commonly assigned U.S. Pat. No. 6,296,638 the complete disclosure of which is incorporated herein by reference. In one embodiment the tip of the electrode (60) presents a relatively narrow surface area, for creating the canals and the defect in the trabecular meshwork, in accordance with the present method.

As the surface area of the tissue treatment surface can vary, and the tissue treatment surface can assume a variety of geometries, the active electrode surface(s) can have area(s) in the range from about 0.25 mm² to 75 mm², usually being from about 0.5 mm² to 40 mm². The geometries can be planar, concave, convex, hemispherical, conical, linear “in-line” array, or virtually any other regular or irregular shape. More commonly, the active electrode(s) or active electrode array(s) will be formed at the distal tip of the electrosurgical instrument shaft, frequently being planar, disk-shaped, pointed or hemispherical surfaces for use in reshaping procedures, or being linear arrays for use in cutting. The active electrode(s) may be formed on lateral surfaces of the electrosurgical instrument shaft (e.g., in the manner of a spatula).

The voltage difference applied between the return electrode(s) and the active electrode is high-frequency voltage or radio frequency voltage, typically between about 5 kHz and 20 MHz, preferably between about 30 kHz and 2.5 MHz, between about 50 kHz and 500 kHz, less than 350 kHz, and between about 100 kHz and 200 kHz. The RMS (root mean square) voltage applied will usually be in the range from about 5 volts to 1000 volts, preferably being in the range from about 10 volts to 500 volts depending on the active electrode size, the operating frequency and the operation mode of the particular procedure or desired effect on the tissue (e.g., contraction, coagulation, cutting or ablation).

A peak-to-peak voltage for ablation or cutting of tissue will be in the range of from about 10 volts to 2000 volts, usually in the range of 200 volts to 1800 volts, and more typically in the range of about 300 volts to 1500 volts, often in the range of about 500 volts to 900 volts peak to peak (again, depending on the electrode size, the operating frequency and the operation mode). Lower peak-to-peak voltages will be used for tissue coagulation or collagen contraction and will typically be in the range from 50 to 1500, preferably from about 100 to 1000, and more preferably from about 120 to 600 volts peak-to-peak

The power source may be current-limited or otherwise controlled so that undesired heating of the target tissue or surrounding (non-target) tissue does not occur. In a preferred embodiment, current-limiting inductors are placed in series with the active electrode where the inductance of the inductor is in the range of 10 microH to 50,000 microH, and depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. A more detailed description of this phenomenon, termed Coblation™, can be found in commonly assigned U.S. Pat. No. 5,683,366 the complete disclosure of which is incorporated herein by reference.

The current flow path between the active electrodes and the return electrode(s) may be generated by submerging the tissue site in an electrically conductive fluid (e.g., body fluid including intra-cellular fluid, a isotonic saline, and an electrically conductive gel), or by directing an electrically conductive fluid through a fluid outlet along a fluid path to the target site (i.e., a liquid, such as isotonic saline, or a gas, such as argon). A conductive gel may also be delivered to the target site to achieve a slower more controlled delivery rate of conductive fluid. In addition, the viscous nature of the gel may allow the surgeon to contain the gel around the target site (e.g., as compared with containment of a liquid, such as isotonic saline). A more complete description of an exemplary method of directing electrically conductive fluid between active and return electrodes is described in U.S. Pat. No. 5,697,281, the contents of which are incorporated by reference herein in their entirety.

With reference to FIG. 5, the present method in one embodiment comprises an electrosurgical procedure for treating glaucoma. In one embodiment, the method (50) includes the steps of: (52) positioning an active electrode in close proximity to the drainage angle, the active electrode disposed on a distal end of a shaft; and (54) applying a high frequency voltage difference between the active electrode and a return electrode sufficient to ablate target tissue in the vicinity of the drainage angle.

In one embodiment, a conductive fluid such as isotonic saline, a conductive gel, and body fluid such as blood, intra cellular fluid, extra-cellular fluid and body plasma is preset and is in contact with the active electrode (68). In this embodiment, the voltage is initially adjusted sufficiently to generate plasma to ablate tissue to form a canal in the trabecular meshwork in treating OAG, and to create a defect with an opening in the iris, in treating NAG. Thereafter the voltage is adjusted to coagulation mode to stabilize the border of the canals, and create self-sealing borders on the defects, to assure prolonged patency of the openings.

In one embodiment, the conductive fluid forms a conductive bridge (68) between the active electrode and the return electrode. In this embodiment, the current does not pass into the tissue, and plasma generated in the conductive fluid is used to modify the tissue as described above.

In an alternative embodiment, an electrically conductive fluid layer is provided in between the active electrode and the tissue, in the vicinity of the tissue. In this embodiment, in addition to plasma generated in the fluid, current from the applied high frequency voltage is applied into the tissue. Thus with this embodiment, both current and plasma are used to modify the tissue. In one embodiment the applied high frequency voltage is adjusted to provide sufficient current for coagulating and sealing the tissue and stop bleeding.

In various embodiments of the method, a suitably configured active electrode is used to treat glaucoma as described herein by ablating and coagulating tissue in the vicinity of the drainage angle and the trabecular meshwork. Thus, for example, an active electrode as schematically illustrated in FIG. 4A and comprised of a narrow distal end, and operating in coblation mode is used to volumetrically remove tissue in the vicinity of the drainage angle. Thereafter, in accordance with the present method, the voltage is switched to coagulation mode to form the stabilized borders in the canals, and the self-sealing borders in the defect on the iris.

In various embodiments, the tissue in the vicinity of the drainage angle is treated with the active electrode for about 0.5 seconds at a time. Also depending on the apparatus used, the conductive fluid is provided by a lumen that discharges the fluid in the vicinity of the tissue. Similarly, in alternate embodiments, a suction lumen is provided to suction fluid and body tissue from the vicinity of the ulcer.

While the invention is described with reference to the Figures and method herein, it will be appreciate by one ordinarily skilled in the art that the invention can also be practiced with modifications within the scope of the claims. The scope of the invention therefore should not be limited to the embodiments as described herein, but is limited only by the scope of the appended claims.

What is claimed is:

1. An electrosurgical method for treating glaucoma, comprising:
positioning an active electrode in close proximity to a drainage angle of the eye, the active electrode disposed on a distal end of a shaft; and
applying a high frequency voltage difference between the active electrode and a return electrode sufficient to ablate target tissue in the vicinity of the drainage angle.

2. The method of claim 1, wherein the high frequency voltage is sufficient to generate plasma between the active and return electrodes.

3. The method of claim 1, wherein an electrically conductive fluid is present at least on the active electrode.

4. The method of claim 3, wherein the electrically conductive fluid is selected from the group consisting of isotonic saline, ringer lactate solution, a conductive gel, intra-cellular body fluid and other conductive body fluid.

5. The method of claim 3, wherein the electrically conductive fluid comprises a conductive fluid bridge between the active and return electrode.

6. The method of claim 3, wherein plasma is generated from the electrically conductive fluid.

7. The method of claim 3, wherein the active electrode is connected to a regulated power supply.

8. The method of claim 3, wherein the electrically conductive fluid is discharged from a lumen integrated with the shaft.

9. The method of claim 3, wherein the high frequency voltage is sufficient to vaporize the electrically conductive fluid.

10. The method of claim 1, wherein the return electrode is prevented from contacting tissue in the vicinity of the drainage angle.

11. The method of claim 1, wherein the active electrode contacts tissue in the vicinity of the drainage angle.

12. The method of claim 1, wherein the shaft is translated axially and radially over the target tissue.

13. The method of claim 1, wherein ablating the target tissue relieves intraocular pressure within the eye.

14. The method of claim 1, wherein ablating the target tissue includes volumetrically removing tissue.

15. The method of claim 1, wherein the target tissue comprises tissue in the iris and the trabecular meshwork.

16. The method of claim 15, wherein ablating the tissue comprise forming a drainage canal in the tissue between the iris and the Schlemm canal in the eye.

17. The method of claim 15, wherein ablating the tissue comprises creating drainage canals within the iris, the canals defined by stabilized borders for resisting scar formation and closure.

18. The method of claim 15, wherein ablating the target tissue comprises forming a defect in the drainage angle, the defect defined by a self-sealing borders for prolonged patency.

19. The method of claim 18, wherein the defect maintains patency between the drainage angle and the Schlemm canal in the eye.

20. The method of claim 19, wherein the defect is from about 0.2 mm to about 0.3 mm in diameter.

21. The method of claim 2, wherein plasma is directed intermittingly to the target tissue for about 0.5 seconds on each instance.

22. The method of claim 1, including adjusting the voltage sufficient to coagulate portions of the target tissue.

23. The method of claim 18, including adjusting the voltage sufficient to coagulate the self-sealing borders.

24. The method of claim 1, wherein the active electrode is selected from a group consisting of a pointed filament electrode, a pointed electrode, a wire electrode, a screen electrode and suction a suction electrode.

25. The method of clam 16, wherein formation of the drainage canals to relieve symptoms of open angle glaucoma.

26. The method of claim 18, wherein formation of the defects relieve the symptoms of narrow angle glaucoma.
